(19) United States
(12) Patent Application Publication
(10) Pub. No.: US 2005/0107456 A1
(43) Pub. Date: May 19, 2005

(54) Substituted pyrazolines for use as pesticides

(76) Inventors: Fritz Maurer, Lauffen (DE); Rainer Fuchs, Wuppertal (DE); Christoph Erdelen, Leichlingen (DE); Angelika Lubos-Erdelen, Leichlingen (DE); Andreas Turberg, Haan (DE)

Correspondence Address: BAYER CROPSCIENCE LP;Patent Department, 100 BAYER ROAD, PITTSBURGH, PA, 15205-9741, US

(21) Appl. No.: 10/501,414

(22) PCT Filed: Jan. 7, 2003
(86) PCT No.: PCT/EP03/00058

(30) Foreign Application Priority Data
Jan. 17, 2002 (DE) 102 01 544.9

(51) Int. Cl. C07D 403/02; A61K 31/416

(52) U.S. Cl. 514/406; 548/365.1

(57) ABSTRACT

The present invention relates to novel substituted pyrazolines of formula (I) 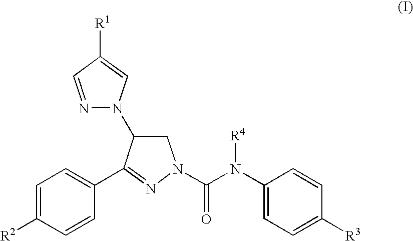
in which R¹, R², R³ and R⁴ are as defined in the disclosure, to a plurality of processes for preparing these compounds and their use for controlling pests, and also to novel intermediates and processes for their preparation.

The present invention relates to novel substituted pyrazolines, to processes for their preparation and to their use as pesticides.

It is known that certain substituted pyrazolines have insecticidal and acaridical properties (cf., for example, DE-A 44 16 112, EP-A 0 679 644 or EP-A 0 438 690). However, in particular at low concentrations of active compound and low application rates, the action of these compounds is not always satisfactory.

This invention provides novel substituted pyrazolines of the formula (I) 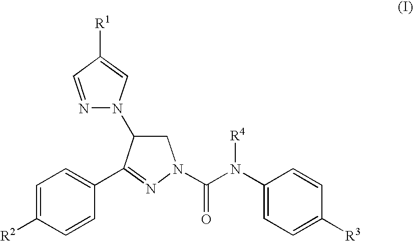
in which
R¹ represents halogen or cyano,
R² represents halogen, haloalkyl, alkoxy, haloalkoxy, alkylthio, haloalkylthio, alkylsulfonyl, haloalkylsulfinyl, haloalkylsulfonyl or cyano,
R³ represents optionally substituted aryl or optionally substituted hetaryl and
R⁴ represents hydrogen, cyanomethyl or alkoxycarbonyl.

Depending on the nature and number of the substituents, the compounds of the formula (I) may be present as geometrical and/or optical isomers, regio isomers and/or configurational isomers or isomer mixtures thereof of varying composition. What is claimed by the invention are both the pure isomers and the isomer mixtures.

Furthermore, it has been found that the substituted pyrazolines of the formula (I) are obtained when
a) pyrazolines of the formula (II) 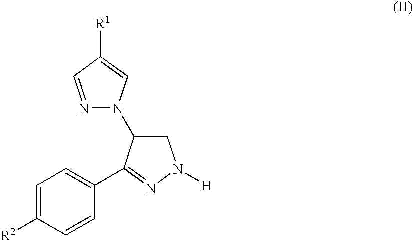
in which
R¹ and R² are as defined above are reacted with isocyanates of the formula (II) 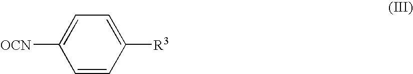
   in which
R³ is as defined above, if appropriate in the presence of a diluent and if appropriate in the presence of a catalyst;
   and
   b) the resulting pyrazoline derivatives of the formula (Ia) according to the invention 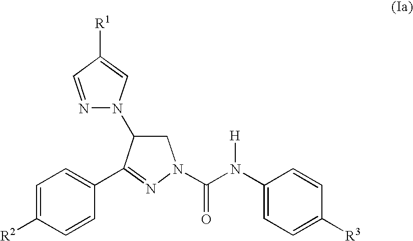
   in which
R¹, R² and R³ are as defined above are optionally reacted with halides of the formula (IV)
   Hal¹-R (IV)
   in which
R⁴ is as defined above and
Hal¹ represents halogen,
   if appropriate in the presence of a diluent and if appropriate in the presence of a base; or
   c) anilines of the formula (V) 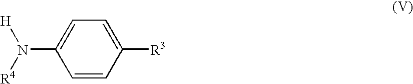
   in which
R³ and R⁴ are as defined above are initially reacted with phosgene in the presence of a diluent and if appropriate in the presence of a base, and the resulting carbamoyl chlorides of the formula (VI) 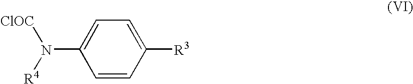
   in which
R³ and R⁴ are as defined above are reacted directly or after intermediate isolation with pyrazolines of the formula (II) 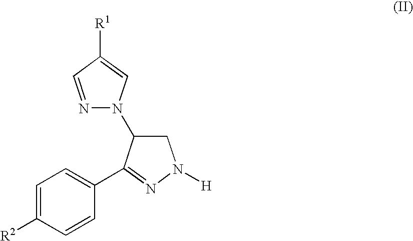
   in which
R¹ and R² are as defined above, in the presence of a diluent and if appropriate in the presence of a base.

Finally, it has been found that the novel substituted pyrazolines of the formula (I) have pronounced biological properties and are suitable especially for controlling animal pests, in particular insects, arachnids and nematodes, encountered in agriculture, in forests, in the protection of stored goods and materials and in the hygiene sector.

The formula (I) provides a general definition of the pyrazoline derivatives according to the invention.

Preferred substituents or ranges of the radicals listed in the formulae above and below are illustrated below:
R¹ preferably represents fluorine, chlorine, bromine, iodine or cyano.
R² preferably represents fluorine, chlorine, bromine, iodine; C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfinyl, C₁-C₄-haloalkylsulfonyl or cyano.
R³ preferably represents aryl which is optionally mono- or polysubstituted by identical or different substituents, examples of substituents which may be mentioned being: halogen, alkyl, alkoxy, alkylthio, alkylsulfonyl, haloalkyl, haloalkoxy, haloalkylthio, haloalkylsulfonyl or cyano;

represents in each case optionally monosubstituted oxadiazolyl or thiadiazolyl, examples of substituents which may be mentioned being: optionally substituted alkyl, optionally substituted alkoxy, optionally substituted alkylthio, optionally substituted aryl or optionally substituted arylalkyl;

represents optionally monosubstituted tetrazolyl, examples of substituents which may be mentioned being: optionally substituted alkyl, optionally substituted alkylthio or alkylsulfonyl, in each case optionally substituted aryl or arylalkyl or optionally substituted cycloalkyl.
R⁴ preferably represents hydrogen, cyanomethyl or C₁-C₄-alkoxycarbonyl.
R¹ particularly preferably represents chlorine, bromine, iodine or cyano.
R² particularly preferably represents fluorine, chlorine, bromine, iodine, cyano, C₁-C₂-alkylthio, C₁-C₂-alkylsulfonyl, and also represents C₁-C₂-haloalkyl, C-C₂-haloalkoxy, C₁-C₂-haloalkylthio or C₁-C₂-haloalkylsulfonyl having in each case 1 to 5 identical or different halogen atoms from the group consisting of fluorine, chlorine and bromine.
R³ particularly preferably represents phenyl which is optionally mono- to trisubstituted by identical or different substituents, examples of substituents which may be mentioned being: fluorine, chlorine, bromine, iodine, cyano; C₁-C₄-alkyl, C₁-C₄-alkoxy, C₁-C₄-alkylthio, C₁-C₄-alkylsulfonyl, and also represents C₁-C₄-haloalkyl, C₁-C₄-haloalkoxy, C₁-C₄-haloalkylthio or C₁-C₄-haloalkylsulfonyl having in each case 1 to 5 identical or different halogen atoms from the group consisting of fluorine, chlorine and bromine;

represents in each case optionally monosubstituted oxadiazolyl or thiadiazolyl, examples of substituents which may be mentioned being: C₁-C₄-alkyl, C₁-C₄-haloalkyl, C₁-C₄-alkoxy, C₁-C₄-haloalkoxy, C₁-C₄-alkylthio, C₁-C₄-haloalkylthio, and also phenyl or benzyl, each of which is optionally mono- to trisubstituted by identical or different substituents from the group consisting of halogen, C₁-C₄-haloalkyl and C₁-C₄-haloalkoxy;

represents optionally substituted tetrazolyl, examples of substituents which may be mentioned being: C₁-C₄-alkyl, C₁-C₄-haloalkyl, C₁-C₄-alkylthio, C₁-C₄-alkylsulfonyl, and also phenyl or benzyl, each of which is optionally mono- to trisubstituted by identical or different substituents from the group consisting of halogen, C₁-C₄-haloalkyl and C₁-C₄-haloalkoxy, furthermore cyclopentyl or cyclohexyl, each of which is optionally mono- to trisubstituted by identical or different substituents from the group consisting of C₁-C₄-alkyl.
R⁴ particularly preferably represents hydrogen, cyanomethyl or C₁-C₄-alkoxycarbonyl.
R¹ very particularly preferably represents chlorine, bromine or cyano.
R² very particularly preferably represents fluorine, chlorine, bromine, iodine, methylthio, trifluoromethyl, trifluoromethoxy or trifluoromethylthio,
R³ very particularly preferably represents phenyl which is optionally mono- to trisubstituted by identical or different substituents, examples of substituents which may be mentioned being: fluorine, chlorine, bromine, iodine, cyano, methyl, methoxy, methylthio, trifluoromethyl, trifluoromethoxy, trifluoromethylthio or trifluoromethylsulfonyl;

represents an oxadiazolyl group from the group consisting of: 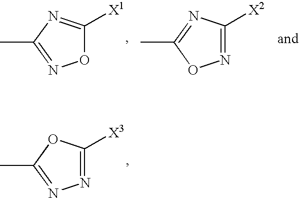
where
X¹, X² and X³ independently of one another represent hydrogen, C₁-C₄-alkyl, C₁-C₄-haloalkyl, C₁-C₄-alkylthio, C₁-C₄-haloalkylthio and also represent phenyl or benzyl, each of which is optionally mono- to trisubstituted by identical or different substituents from the group consisting of halogen, C₁-C₂-haloalkyl or C₁-C₂-haloalkoxy having in each case 1 to 3 identical or different halogen atoms from the group consisting of fluorine, chlorine and bromine;

represents a tetrazolyl group from the group consisting of: 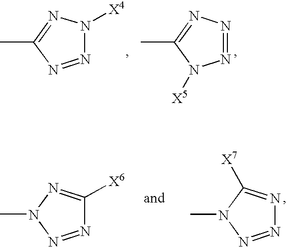
where
X⁴, X⁵, X⁶ and X⁷ independently of one another represent hydrogen, C₁-C₄-alkyl, C₁-C₂-haloalkyl having 1 to 3 identical or different halogen atoms from the group consisting of fluorine, chlorine and bromine; C₁-C₄-alkylthio, C₁-C₄-alkylsulfonyl, represent phenyl or benzyl, each of which is optionally mono- to trisubstituted by identical or different substituents from the group consisting of halogen, C₁-C₂-haloalkyl and C₁-C₂-haloalkoxy having in each case 1 to 3 identical or different halogen atoms from the group consisting of fluorine, chlorine and bromine; and also represent cyclopentyl or cyclohexyl, each of which is optionally mono- to trisubstituted by C₁-C₄-alkyl.
R⁴ very particularly preferably represents hydrogen, cyanomethyl, methoxycarbonyl, ethoxycarbonyl, n- or i-propoxycarbonyl, n-, i-, s- or t-butoxycarbonyl.
R¹ especially preferably represents chlorine or cyano.
R² especially preferably represents fluorine, chlorine, bromine, iodine or trifluoromethylthio.
R³ especially preferably represents phenyl which is optionally mono- or disubstituted by identical or different substituents from the group consisting of fluorine, chlorine, trifluoromethyl, trifluoromethoxy and trifluoromethylthio;

represents an oxadiazolyl group from the group consisting of: 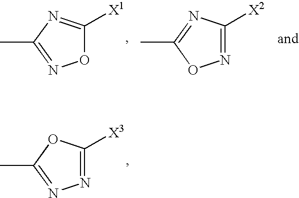
where
X¹, X² and X³ independently of one another represent hydrogen, methyl, ethyl, n- or i-propyl, n-, i-, s- or t-butyl, trifluoromethyl, trifluoromethoxy or trifluoromethylthio and also represent phenyl or benzyl, each of which is optionally mono- or disubstituted by identical or different substituents from the group consisting of fluorine, chlorine, bromine, trifluoromethyl or trifluoromethoxy;

represents a tetrazolyl group from the group consisting of: 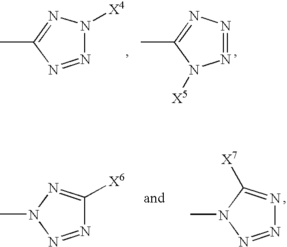
where
X⁴, X⁵, X⁶ and X⁷ independently of one another represent hydrogen, methyl, ethyl, n- or i-propyl, n-, i-, s- or t-butyl; fluoromethyl, difluoromethyl, trifluoromethyl, 1,1-difluoroethyl, 2,2,2-trifluoroethyl, methylthio, ethylthio, methylsulfonyl, ethylsulfonyl; represent phenyl or benzyl, each of which is optionally mono- to disubstituted by identical or different substituents from the group consisting of fluorine, chlorine, bromine, methyl, methoxy, trifluoromethyl and trifluoromethoxy; and also represent cyclohexyl which is optionally mono- to disubstituted by methyl.
R⁴ especially preferably represents hydrogen or cyanomethyl.

Preference is furthermore given to compounds of the formula (I) in which R¹ represents cyano.

Preference is furthermore given to compounds of the formula (I) in which R² represents halogen, preferably fluorine, chlorine, bromine, iodine, particularly preferably fluorine or chlorine, very particularly preferably chlorine.

Preference is furthermore given to compounds of the formula (I) in which R¹ represents cyano and R² represents chlorine.

Preference is furthermore given to compounds of the formula (I) in which R⁴ represents hydrogen or cyanomethyl.

Preference is furthermore given to compounds of the formula (I) in which R³ represents phenyl which is optionally mono- to trisubstituted by identical or different substituents, examples of substituents which may be mentioned being: fluorine, chlorine, bromine, iodine, cyano; C₁-C₄-alkyl, C₁-C₄-alkoxy, C₁-C₄-alkylthio, C₁-C₄-alkylsulfonyl, and also represents C₁-C₄-haloalkyl, C₁-C₄-haloalkoxy, C₁-C₄-haloalkylthio or C₁-C₄-haloalkylsulfonyl having in each case 1 to 5 identical or different halogen atoms from the group consisting of fluorine, chlorine and bromine; preferably represents phenyl which is optionally mono- to trisubstituted by identical or different substituents, examples of substituents which may be mentioned being: fluorine, chlorine, bromine, iodine, cyano, methyl, methoxy, methylthio, trifluoromethyl, trifluoromethoxy, trifluoromethylthio or trifluoromethylsulfonyl; particularly preferably represents phenyl which is optionally mono- or disubstituted by identical or different substituents from the group consisting of fluorine, chlorine, trifluoromethyl, trifluoromethoxy and trifluoromethylthio.

Preference is furthermore given to compounds of the formula (I) in which R³ represents oxadiazolyl or thiadiazolyl, each of which is optionally monosubstituted, examples of substituents which may be mentioned being: C₁-C₄-alkyl, C₁-C₄-haloalkyl, C₁-C₄-alkoxy, C₁-C₄-haloalkoxy, C₁-C₄-alkylthio, C₁-C₄-haloalkylthio, and also phenyl or benzyl, each of which is optionally mono- to trisubstituted by identical or different substituents from the group consisting of halogen, C₁-C₄-haloalkyl and C₁-C₄-haloalkoxy;

preferably represents an oxadiazolyl group from the group consisting of: 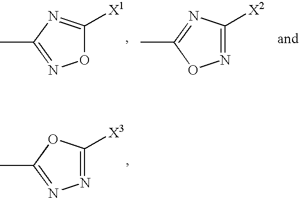
where
X¹, X² and X³ independently of one another represent hydrogen, C₁-C₄-alkyl, C₁-C₄-haloalkyl, C₁-C₄-alkylthio, C₁-C₄-haloalkylthio, and also represent phenyl or benzyl, each of which is optionally mono- to trisubstituted by identical or different substituents from the group consisting of halogen, C₁-C₂-haloalkyl or C₁-C₂-haloalkoxy having in each case 1 to 3 identical or different halogen atoms from the group consisting of fluorine, chlorine and bromine;

particularly preferably represents an oxadiazolyl group from the group consisting of: 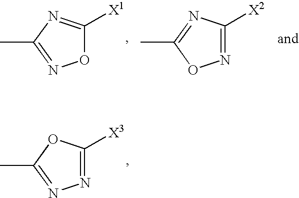
where
X¹, X² and X³ independently of one another represent hydrogen, methyl, ethyl, n- or i-propyl, n-, i-, s- or t-butyl, trifluoromethyl, trifluoromethoxy or trifluoromethylthio, and also represent phenyl or benzyl, each of which is optionally mono- or disubstituted by identical or different substituents from the group consisting of fluorine, chlorine, bromine, trifluoromethyl and trifluoromethoxy.

Preference is furthermore given to compounds of the formula (I) in which R³ represents optionally substituted tetrazolyl, substituents which may be mentioned being: C₁-C₄-alkyl, C₁-C₄-haloalkyl, C₁-C₄-alkylthio, C₁-C₄-alkylsulfonyl, and also phenyl or benzyl, each of which is optionally mono- to trisubstituted by identical or different substituents from the group consisting of halogen, C₁-C₄-haloalkyl and C₁-C₄-haloalkoxy, furthermore cyclopentyl or cyclohexyl, each of which is optionally mono- to trisubstituted by identical or different substituents from the group consisting of C₁-C₄-alkyl;

preferably represents a tetrazolyl group from the group consisting of: 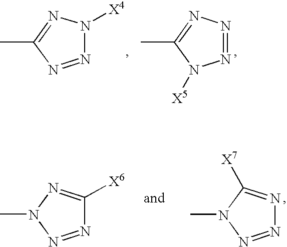
where
X⁴, X⁵, X⁶ and X⁷ independently of one another represent hydrogen, C₁-C₄-alkyl, C₁-C₂-haloalkyl having 1 to 3 identical or different halogen atoms from the group consisting of fluorine, chlorine and bromine; C₁-C₄-alkylthio, C₁-C₄-alkylsulfonyl, represent phenyl or benzyl, each of which is optionally mono- to trisubstituted by identical or different substituents from the group consisting of halogen, C₁-C₂-haloalkyl and C₁-C₂-haloalkoxy having in each case 1 to 3 identical or different halogen atoms from the group consisting of fluorine, chlorine and bromine; and also represent cyclopentyl or cyclohexyl, each of which is optionally mono- to trisubstituted by C₁-C₄-alkyl;

particularly preferably represents a tetrazolyl group from the group consisting of: 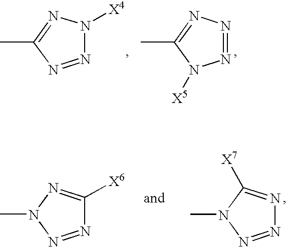
where
X⁴, X⁵, X⁶ and X⁷ independently of one another represent hydrogen, methyl, ethyl, n- or i-propyl, n-, i-, s- or t-butyl; fluoromethyl, difluoromethyl, trifluoromethyl, 1,1-difluoroethyl, 2,2,2-trifluoroethyl, methylthio, ethylthio, methylsulfonyl, ethylsulfonyl; represent phenyl or benzyl, each of which is optionally mono- to disubstituted by identical or different substituents from the group consisting of fluorine, chlorine, bromine, methyl, methoxy, trifluoromethyl and trifluoromethoxy; and also represent cyclohexyl which is optionally mono- to disubstituted by methyl.

The radical definitions or illustrations listed above or in preferred ranges apply to the end products and correspondingly to starting materials and intermediates. These radical definitions can be combined with one another as desired, i.e. including combinations between the respective preferred ranges.

Preference according to the invention is given to the compounds of the formula (I) which contain a combination of the meanings listed above as being preferred (preferable).

Particular preference according to the invention is given to the compounds of the formula (I) which contain a combination of the meanings listed above as being particularly preferred.

Very particular preference according to the invention is given to the compounds of the formula (I) which contain a combination of the meanings listed above as being very particularly preferred.

Special preference according to the invention is given to the compounds of the formula (I) which contain a combination of the meanings listed above as being especially preferred.

In the radical definitions given above and below, hydrocarbon radicals, such as alkyl, are in each case straight-chain or branched as far as this is possible—including in combination with heteroatoms such as alkoxy.

Using, for example, 3-(4-chlorophenyl)-4-(4-cyanopyrazol-1-yl)-4,5-dihydro-1H-pyrazole and 4-biphenyl isocyanate as starting materials, the course of the reaction of the process (a) according to the invention can be represented by the formula scheme below:

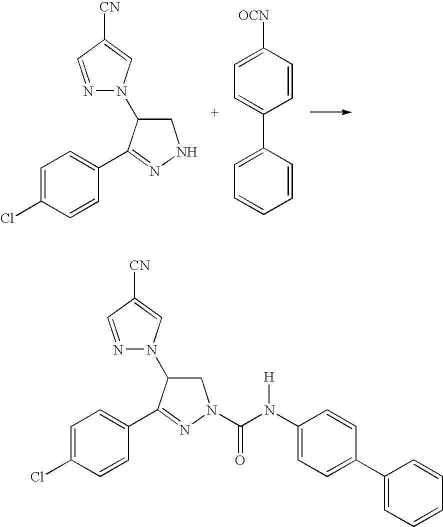

Using, for example, N-(4-phenyl)-3-(4-chlorophenyl)-4-(4-cyanopyrazol-1-yl)-4,5-di-hydro-1-pyrazolecarboxanilide and bromacetonitrile as starting materials, the course of the reaction of the process (b) according to the invention can be represented by the formula scheme below:

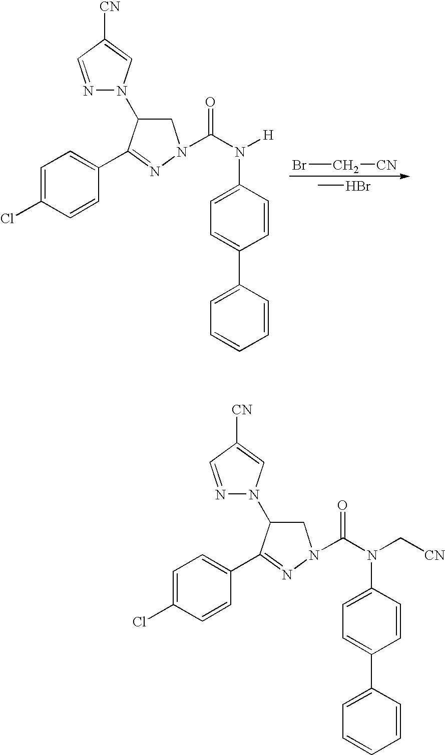

Using, for example, 4-(5-tert-butyl-1,2,4-oxadiazol-3-yl)-aniline, triphosgene (bis(trichloromethyl) carbonate) and 3-(4-chlorophenyl)-4-(4-cyanopyrazol-1-yl)-4,5-dihydro-1H-pyrazole as starting materials, the course of the reaction of the process (c) according to the invention can be represented by the formula scheme below:

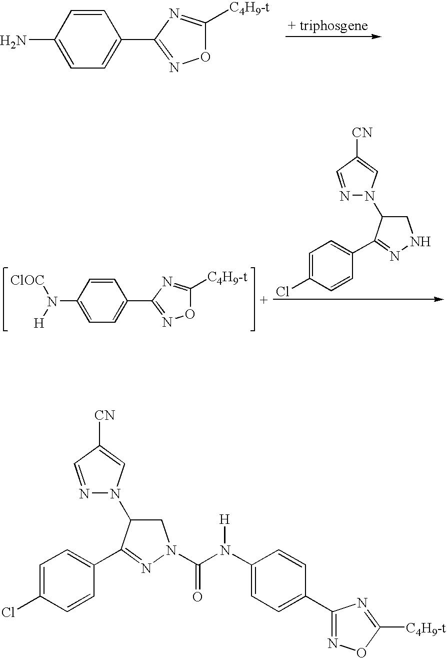

The formula (II) provides a general definition of the pyrazolines to be used as starting materials for carrying out the processes (a) and (c) according to the invention. In this formula, R¹ and R² preferably, particularly preferably, very particularly preferably and especially preferably have those meanings which have already been mentioned in connection with the description of the compounds of the formula (I) according to the invention as being preferred, particularly preferred, very particularly preferred and especially preferred for these radicals.

Some of the pyrazolines of the formula (II) are known (cf. for example, EP 0 438 690); some form part of the subject matter of DE 101 35 551 (dated Jul. 20, 2001).

Pyrazolines of the formula (II) are obtained when substituted acetophenones of the formula (VII) 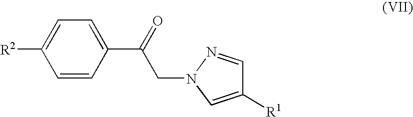
in which
R¹ and R² are as defined above are, in a first step, reacted with known bisdialkylaminomethanes of the formula (VIII)
   (Alk)₂N—CH₂—N(Alk)₂ (VIII)
   in which
alk represents C₁-C₄-alkyl in the presence of an inert organic solvent (preferably halogenated hydrocarbons, such as, for example, methylene chloride or ethylene chloride) at temperatures between 0° C. and 120° C., preferably between 20° C. and 80° C. (cf., for example, EP-A 0 546 420), and the resulting dialkylaminoalkyl ketones of the formula (IX) 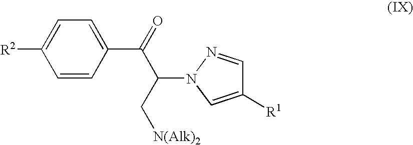
   in which
R¹, R² and Alk are as defined above are, if required, isolated and, in a second step, reacted with hydrazine (hydrate) in the presence of an inert organic solvent (preferably alcohols, such as, for example, methanol or ethanol) at temperatures between 0° C. and 80° C., preferably between 20° C. and 50° C. (cf. also the Preparation Examples).

The substituted acetophenones of the formula (VII) are obtained when haloacetophenones of the formula (X) 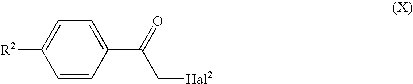
in which
R² is as defined above and
Hal² represents halogen are reacted with pyrazoles of the formula (XI) 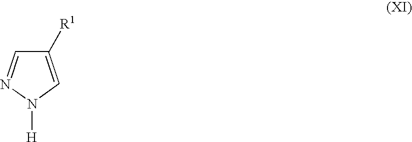
   in which
R¹ is as defined above in the presence of an inorganic or organic base (for example potassium carbonate) and, if appropriate, in the presence of an inert organic solvent (for example acetonitrile), at temperatures between 0° C. and 100° C., preferably between 20° C. and 80° C. (cf., for example, EP-A 0 438 690 and also the Preparation Examples).

The haloacetophenones of the formula (X) and the pyrazoles of the formula (XI) are generally known compounds of organic chemistry and/or can be obtained in a generally known manner.

The formula (III) provides a general definition of the isocyanates further to be used as starting materials in the process (a) according to the invention. In this formula, R³ preferably, particularly preferably, very particularly preferably and especially preferably has those meanings which have already been mentioned in connection with the description of the compounds of the formula (I) according to the invention as being preferred, particularly preferred, very particularly preferred and especially preferred for this radical.

The isocyanates of the formula (III) are generally known compounds of organic chemistry and/or can be obtained in a generally known manner.

The formula (IV) provides a general definition of the halides to be used as starting materials for carrying out the process (b) according to the invention. In this formula, R⁴ preferably, particularly preferably, very particularly preferably and especially preferably has those meanings which have already been mentioned in connection with the description of the compounds of the formula (I) according to the invention as being preferred, particularly preferred, very particularly preferred and especially preferred for this radical. Hal¹ preferably represents chlorine or bromine.

The halides of the formula (IV) are generally known compounds of organic chemistry.

The formula (V) provides a general definition of the anilines to be used as starting materials for carrying out the process (c) according to the invention. In this formula, R³ and R⁴ preferably, particularly preferably, very particularly preferably and especially preferably have those meanings which have already been mentioned in connection with the description of the compounds of the formula (I) according to the invention as being preferred, particularly preferred, very particularly preferred and especially preferred for these radicals.

Most of the anilines of the formula (V) are known (cf., for example, U.S. Pat. No. 3,270,029, U.S. Pat. No. 3,793,340, U.S. Pat. No. 3,810,901, WO 97/14695, WO 98/28269, WO 98/50358, EP 0 155 507, EP 0 933 581, DE 100 23 430; J. Med. Chem. 1978, 21, 1093-1100; Zh. Org. Khim. 1976, 12, 1054-1057; Can. J. Chem. 1998, 76, 78-84 and J. Chem. Soc., C. Org. 1966, 840-845); some of them also form part of the subject matter of DE 101 39 721 (dated Aug. 13, 2001), and/or they can be obtained in a known manner.

Thus, for example, the 4-(1,2,4-oxadiazol-3-yl)anilines of the formula (Va) 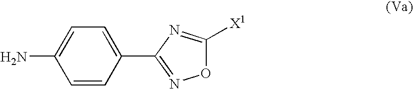
in which
X¹ is as defined above are obtained when 4-aminobenzamidoxime of the formula (XII) 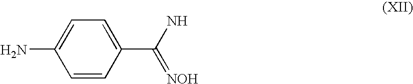
   is reacted with an acylating agent of the formula (XIII)
   X¹—CO-A (XIII)
   in which
A represents a customary leaving group, such as, in particular, chlorine, —OC₁-C₄-alkyl or —OCOX¹, and
X¹ is as defined above, if appropriate in the presence of an inert organic solvent (for example ethanol) and if appropriate in the presence of a base (for example alkali metal alkoxides) at temperatures between 20° C. and 120° C., preferably between 50° C. and 100° C. (cf. also the Preparation Examples).

The 4-aminobenzamidoxime of the formula (XII) is obtained in a generally known manner when 4-cyanoaniline is reacted with hydroxylamine hydrochloride in the presence of a diluent, such as, preferably, aliphatic alcohols, and in the presence of a base (for example potassium carbonate) at temperatures between 20° C. and 120° C., preferably between 50° C. and 100° C.

Furthermore, for example, the 4-(1,2,4-oxadiazol-5-yl)anilines of the formula (Vb) 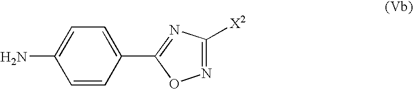
in which
X² is as defined above can be obtained when 4-aminobenzoic acid esters of the formula (XIV) 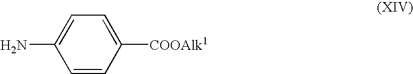
   in which
alk¹ represents C₁-C₄-alkyl are reacted with amidoximes of the formula (XV) 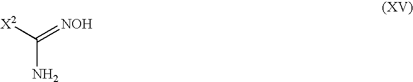
   in which
X² is as defined above, if appropriate in the presence of an inert organic solvent (such as, preferably, aliphatic alcohols) and if appropriate in the presence of a base (for example alkali metal alkoxides) at temperatures between 20° C. and 120° C., preferably between 50° C. and 100° C. (cf. also the Preparation Examples).

Furthermore, for example, 4-(tetrazol-5-yl)anilines of the formulae (Vc) and (Vd) 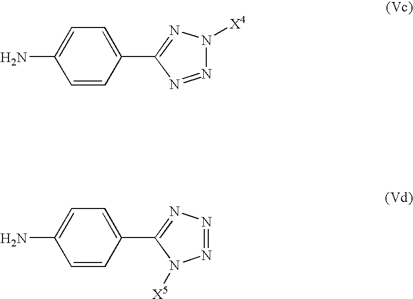
in which
X⁴ and X⁵ are as defined above can be obtained when aminobenzonitrile of the formula (XVI) 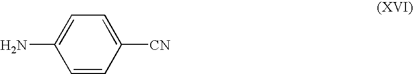
   is reacted with sodium azide and, for example, triethylamine hydrochloride in the presence of an inert organic solvent (for example acetonirile, dimethylformamide or toluene), preferably under reflux, and the resulting tetrazoleanilines of the formula (V-A) 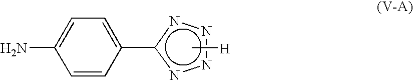
   are, if appropriate, reacted with compounds of the formula (XVI)
   E-X (XVI)
   in which
X has the meanings given above for X⁴ and X⁵, except for hydrogen, and
E represents an anionic leaving group, such as, preferably, chlorine, bromine, iodine, acetoxy, tosyl or mesyl, in the presence of a diluent (for example acetonitrile or dimethylformamide) and, if appropriate, in the presence of an acid acceptor (for example potassium carbonate) at temperatures between 0° C. and 80° C., preferably between 20° C. and 50° C.

When preparing compounds in which X represents a tertiary alkyl radical, it is in some cases found to be advantageous to react the compounds of the formula (V-A) with tertiary alcohols in the presence of strong acids (for example trifluoroacetic acid and/or sulfuric acid) (cf. also the Preparation Examples).

Furthermore, for example, 4-(tetrazol-2-yl)anilines of the formula (Ve) 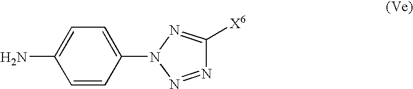
in which
X⁶ is as defined above can be obtained when 4-(tetrazol-2-yl)nitrobenzenes of the formula (XVII) 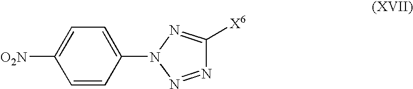
   in which
X⁶ is as defined above are reacted with customary reducing agents (for example hydrazine hydrate), if appropriate in the presence of a catalyst (for example iron(III) chloride) in the presence of an inert organic solvent (for example methanol or tetrahydrofuran) at temperatures between 50° C. and 120° C. (cf. also the Preparation Examples).

The 4-(tetrazol-2-yl)nitrobenzenes of the formula (XVII) can be obtained, for example, when 4-fluoronitrobenzene of the formula (XVIII) 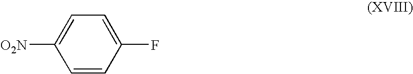
is reacted with tetrazoles of the formula (XIX) 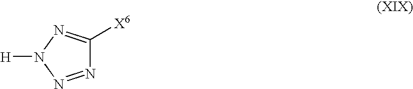
in which
X⁶ is as defined above in a known manner, if appropriate in the presence of an inert organic solvent (for example dimethylformamide) and in the presence of a base (for example potassium carbonate) at temperatures between 20° C. and 150° C., preferably between 50° C. and 120° C. (cf. EP 0 884 311 and the Preparation Examples).

The process (a) according to the invention is preferably carried out using diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroine, benzene, toluene, xylene, methylene chloride, ethylene chloride, chloroform, carbon tetrachloride, chlorobenzene and o-dichlorobenzene, ethers, such as diethyl ether and dibutyl ether, methyl tert-butyl ether, methyl tert-amyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methylpyrrolidone, and also dimethyl sulfoxide, tetramethylene sulfone or hexamethylphosphoric triamide.

The process (a) according to the invention is preferably also carried out using a catalyst. Suitable catalysts are in particular tertiary organic amines, such as, for example, triethylamine.

In the process (a) according to the invention, the reaction temperatures can be varied within a relatively wide range. In general, the process is carried out at temperatures between 0° C. and 120° C., preferably at temperatures between 20° C. and 80° C.

The process (a) according to the invention is generally carried out under atmospheric pressure. However, it is also possible to operate under elevated or reduced pressure.

For carrying out the process (a) according to the invention, the starting materials are generally employed in approximately equimolar amounts. However, it is also possible to use a relatively small excess of one of the two components employed. Work-up is carried out by customary methods (cf. the Preparation Examples).

The processes (b) and (c) according to the invention are preferably carried out using diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroine, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methylpyrrolidone, and also dimethyl sulfoxide, tetramethylene sulfone or hexamethylphosphoric triamide.

Suitable bases for carrying out the processes (b) and (c) according to the invention are all acid binders which can be customarily employed for such reactions. Preference is given to using alkali metal and alkaline earth metal hydrides, such as lithium hydride, sodium hydride, potassium hydride or calcium hydride; alkali metal and alkaline earth metal hydroxides, such as lithium hydroxide, sodium hydroxide, potassium hydroxide or calcium hydroxide; alkali metal and alkaline earth metal carbonates or bicarbonates, such as sodium carbonate or potassium carbonate or sodium bicarbonate or potassium bicarbonate or calcium carbonate; alkali metal acetates, such as sodium acetate or potassium acetate, alkali metal alkoxides, such as sodium tert-butoxide or potassium tert-butoxide; furthermore basic nitrogen compounds, such as trimethylamine, triethylamine, tripropylamine, tributylamine, diisobutylamine, dicyclohexylamine, ethyldiisopropylamine, ethyldicyclohexylamine, N,N-dimethylbenzylamine, N,N-dimethylaniline, pyridine, 2-methyl-, 3-methyl-, 4-methyl-, 2,4-dimethyl-, 2,6-dimethyl-, 2-ethyl-, 4-ethyl- and 5-ethyl-2-methylpyridine, 1,5-diazabicyclo[4.3.0]-non-5-ene (DBN), 1,8-diazabicyclo[5.4.0]-undec-7-ene (DBU), 1,4-diazabicyclo[2.2.2]-octane (DABCO).

When carrying out the process (b) according to the invention, the reaction temperatures can be varied within a relatively wide range. In general, the process is carried out at temperatures between 0° C. and 120° C., preferably at temperatures between 20° C. and 80° C.

The process (b) according to the invention is generally carried out under atmospheric pressure. However, it is also possible to operate under elevated or reduced pressure.

To carry out the process (b) according to the invention, the starting materials are generally employed in approximately equimolar amounts. However, it is also possible to use an excess of halide and base. Work-up is carried out by customary methods (cf the Preparation Examples).

When carrying out the process (c) according to the invention, the reaction temperatures can be varied within a relatively wide range. In general, the process is carried out at temperatures between −10° C. and 120° C., preferably at temperatures between 0° C. and 100° C.

The process (c) according to the invention is generally carried out under atmospheric pressure. However, it is also possible to operate under elevated or reduced pressure.

To carry out the process (c) according to the invention, the starting materials are generally employed in approximately equimolar amounts. However, it is also possible to use an excess of chloride and base. Work-up is carried out by customary methods (cf. the Preparation Examples).

The active compounds, having good plant tolerance and favorable warm-blood toxicity, are suitable for controlling animal pests, in particular insects, arachnids and nematodes, which are encountered in agriculture, in forestry, in the protection of stored products and of materials, and in the hygiene sector. They may be preferably used as crop protection agents. They are active against normally sensitive and resistant species and against all or some stages of development. The abovementioned pests include:
From the order of the Isopoda, for example, Oniscus asellus, Armadillidium vulgare and Porcellio scaber.
From the order of the Diplopoda, for example, Blaniulus guttulatus.
From the order of the Chilopoda, for example, Geophilus carpophagus and Scutigera spp.
From the order of the Symphyla, for example, Scutigerella immaculata.
From the order of the Thysanura, for example, Lepisma saccharina.
From the order of the Collembola, for example, Onychiurus armatus.
From the order of the Orthoptera, for example, Acheta domesticus, Gryllotalpa spp., Locusta migratoria migratorioides, Melanoplus spp. and Schistocerca gregaria.
From the order of the Blattaria, for example, Blatta orientalis, Periplaneta americana, Leucophaea maderae and Blattella germanica.
From the order of the Dermaptera, for example, Forficula auricularia.
From the order of the Isoptera, for example, Reticulitermes spp.
From the order of the Phthiraptera, for example, Pediculus humanus corporis, Haematopinus spp., Linognathus spp., Trichodectes spp. and Damalinia spp.
From the order of the Thysanoptera, for example, Hercinothrips femoralis, Thrips tabaci, Thrips palmi and Frankliniella accidentalis.
From the order of the Heteroptera, for example, Eurygaster spp., Dysdercus intermedius, Piesma quadrata, Cimex lectularius, Rhodnius prolixus and Triatoma spp.
From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.
From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofinannophila pseudospretella, Cacoecia podana, Capua reticulana, Choristoneura fumiferana, Clysia ambiguella, Homona magnanima, Tortrix viridana, Cnaphalocerus spp. and Oulema oryzae.
From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachria varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.
From the order of the Hymenoptera, for example, Diprion spp., Hoplocampa spp., Lasius spp., Monomorium pharaonis and Vespa spp.
From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.
From the order of the Siphonaptera, for example, Xenopsylla cheopis and Ceratophyllus spp.
From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

The compounds of the formula (I) according to the invention can be employed with particularly good results for controlling plant-damaging insects, such as, for example, against the larvae of the mustard beetle (Phaedon cochleariae) or the caterpillars of the army worm (Spodoptera frugiperda).

At certain concentrations or application rates, the compounds according to the invention may, if appropriate, also be used as herbicides and microbicides, for example as fungicides, antimycotics and bactericides. If appropriate, they may also be used as intermediates or precursors for the synthesis of further active compounds.

All plants and plant parts can be treated in accordance with the invention. Plants are to be understood as meaning in the present context all plants and plant populations such as desired and undesired wild plants or crop plants (inclusive of naturally occurring crop plants). Crop plants can be plants which can be obtained by conventional plant breeding and optimization methods or by biotechnological and recombinant methods or by combinations of these methods, inclusive of the transgenic plants and inclusive of the plant cultivars protectable or not protectable by plant breeders' rights. Plant parts are to be understood to mean all above-ground and underground parts and organs of plants, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted into the customary formulations, such as solutions, emulsions, wettable powders, suspensions, powders, dusts, pastes, soluble powders, granules, suspension-emulsion concentrates, natural and synthetic materials impregnated with active compound, and microencapsulations in polymeric substances.

These formulations are produced in a known manner, for example by mixing the active compounds with extenders, that is liquid solvents and/or solid carriers, optionally with the use of surfactants, that is emulsifiers and/or dispersants, and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulfoxide, and also water.

Suitable solid carriers are: for example ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and also protein hydrolysates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic dyes, such as alizarin dyes, azo dyes and metal phthalocyanine dyes, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention, as such or in their formulations, can also be used as a mixture with known fungicides, bactericides, acaricides, nematicides, or insecticides, for example to widen the spectrum of activity or to prevent the development of resistance. In many cases, synergistic effects are achieved i.e., the efficacy of the mixture is greater than the efficacy of the individual components.

Suitable mixing partners are, for example, the following compounds:

Fungicides:

aldimorph, ampropylfos, ampropylfos-potassium, andoprim, anilazine, azaconazole, azoxystrobin, benalaxyl, benodanil, benomyl, benzamacril, benzamacryl-isobutyl, bialaphos, binapacryl, biphenyl, bitertanol, blasticidin-S, bromuconazole, bupirimate, buthiobate, calcium polysulfide, carpropamide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon, ediphenphos, epoxiconazole, etaconazole, ethirimol, etridiazole, famoxadon, fenapanil, fenarimol, fenbuconazole, fenfuram, fenhexamide, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminum, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole, imazalil, imibenconazole, iminoctadine, iminoctadine albesilate, iminoctadine triacetate, iodocarb, ipconazole, iprobenfos (IBP), iprodione, iprovalicarb, irumamycin, isoprothiolane, isovaledione, kasugamycin, kresoxim-methyl, copper preparations, such as: copper hydroxide, copper naphthenate, copper oxychloride, copper sulfate, copper oxide, oxine-copper and Bordeaux mixture, mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur, quinconazole, quintozene (PCNB), quinoxyfen, sulfur and sulfur preparations, spiroxamines, tebuconazole, tecloftalam, tecnazene, teteyclacis, tetraconazole, thiabendazole, thicyofen, thifluzamides, thiophanate-methyl, thiram, tioxymid, tolclofos-methyl, tolylfluanid, triadimefon, triadimenol, triazbutil, triazoxide, trichlamide, tricyclazole, tridemorph, trifloxystrobins, triflumizole, triforine, triticonazole, uniconazole, validamycin A, vinclozolin, viniconazole, zarilamide, zineb, ziram and also Dagger G, OK-8705, OK-8801, α-( 1,1 -dimethylethyl)-β-(2-phenoxyethyl)-1H-1,2,4-triazole-1-ethanol, α-(2,4-dichlorophenyl)-β-fluoro-β-propyl-1H-1,2,4-triazole-1-ethanol, α-(2,4-dichlorophenyl)-β-methoxy-α-methyl-1H-1,2,4-triazole-1-ethanol, α-(5-methyl-1,3-dioxan-5-yl)-β-[[4-(trifluoromethyl)-phenyl]-methylene]-1H-1,2,4-triazole-1-ethanol, (5RS,6RS)-6-hydroxy-2,2,7,7-tetramethyl-5-(1H-1,2,4-triazol-1-yl)-3-octanone, (E)-a-(methoxyimino)-N-methyl-2-phenoxy-phenylacetamide, 1-(2,4-dichlorophenyl)-2-(1H-1,2,4-triazol-1-yl)-ethanone-O-(phenylmethyl)-oxime, 1-(2-methyl-1-naphthalenyl)-1H-pyrrole-2,5-dione, 1-(3,5-dichlorophenyl)-3-(2-propenyl)-2,5-pyrrolidinedione, 1-[(diiodomethyl)-sulfonyl]-4-methyl-benzene, 1-[[2-(2,4-dichlorophenyl)-1,3-dioxolan-2-yl]-methyl]-1H-imidazole, 1-[[2-(4-chlorophenyl)-3-phenyloxiranyl]-methyl]-1H-1,2,4-triazole, 1-[1-[2-[(2,4-dichlorophenyl)-methoxy]-phenyl]-ethenyl]-1H-imidazole, 1-methyl-5-nonyl-2-(phenylmethyl)-3-pyrrolidinol, 2′,6′-dibromo-2-methyl-4′-trifluoromethoxy-4′-trifluoro-methyl-1,3-thiazole-5-carboxanilide, 2,6-dichloro-5-(methylthio)-4-pyrimidinyl-thiocyanate, 2,6-dichloro-N-(4-trifluoromethylbenzyl)-benzamide, 2,6-dichloro-N-[[4-(trifluoromethyl)-phenyl]-methyl]-benzamide, 2-(2,3,3-triiodo-2-propenyl)-2H-tetrazole, 2-[(1 -methylethyl)-sulfonyl]-5-(trichloromethyl)-1,3,4-thiadiazole, 2-[[6-deoxy-4-O-(4-O-methyl-β-D-glycopyranosyl)-α-D-glucopyranosyl]-amino]-4-methoxy-1H-pyrrolo[2,3-d]pyrimidine-5-carbonitrile, 2-aminobutane, ²-bromo-2-(bromomethyl)-pentanedinitrile, 2-chloro-N-(2,3-dihydro-1,1,3-trimethyl-1H-inden-4-yl)-3-pyridinecarboxamide, 2-chloro-N-(2,6-dimethylphenyl)-N-(isothiocyanatomethyl)-acetarnide, 2-phenylphenol (OPP), 3,4-dichloro-1-[4-(difluoromethoxy)-phenyl]-1H-pyrrole-2,5-dione, 3,5-dichloro-N-[cyano-[(1-methyl-2-propynyl)-oxy]-methyl]-benzamide, 3-(1,1-dimethylpropyl)-1-oxo-1H-indene-2-carbonitrile, 3-[2-(4-chlorophenyl)-5-ethoxy-3-isoxazolidinyl]-pyridine, 4-chloro-2-cyano-N,N-dimethyl-5-(4-methylphenyl)-1H-imidazole-1-sulfonamide, 4-methyl-tetrazolo[1,5-a]quinazolin-5(4H)-one, 8-hydroxyquinoline sulfate, 9H-xanthene-2-[(phenylamino)-carbonyl]-9-carboxylic hydrazide, bis-(1-methylethyl)-3-methyl-4-[(3-methylbenzoyl)-oxy]-2,5-thiophenedicarboxylate, cis-1-(4-chlorophenyl)-2-(1H-1,2,4-triazol-1-yl)-cycloheptanol, cis-4-[3-[4-(1,1 -dimethylpropyl)-phenyl-2-methylpropyl]-2,6-dimethyl-morpholinehydrochloride, ethyl [(4-chlorophenyl)-azo]-cyanoacetate, potassium hydrogen carbonate, methanetetrathiol sodium salt, methyl 1-(2,3-dihydro-2,2-dimethyl-1H-inden-1-yl)-1H-imidazole-5-carboxylate, methyl N-(2,6-dimethylphenyl)-N-(5-isoxazolylcarbonyl)-DL-alaninate, methyl N-(chloroacetyl)-N-(2,6-dimethylphenyl)-DL-alaninate, N-(2,6-dimethylphenyl)-2-methoxy-N-(tetrahydro-2-oxo-3 -furanyl)-acetamide, N-(2,6-dimethylphenyl)-2-methoxy-N-(tetrahydro-2-oxo-3 -thienyl)-acetamide, N-(2-chloro-4-nitrophenyl)-4-methyl-3-nitro-benzenesulfonamide, N-(4-cyclohexylphenyl)-1,4,5,6-tetrahydro-2-pyrimidinamine, N-(4-hexylphenyl)-1,4,5,6-tetrahydro-2-pyrimidinamine, N-(5-chloro-2-methylphenyl)-2-methoxy-N-(2-oxo-3-oxazolidinyl)-acetamide, N-(6-methoxy-3 -pyridinyl)-cyclopropanecarboxamide, N-[2,2,2-trichloro-1-[(chloroacetyl)-amino]-ethyl]-benzamide, N-[3-chloro-4,5-bis-(2-propinyloxy)-phenyl]-N′-methoxy-methanimidamide, N-formyl-N-hydroxy-DL-alanine sodium salt, O,O-diethyl [2-(dipropylamino)-2-oxoethyl]-ethylphosphoramidothioate, O-methyl S-phenyl phenylpropylphosphoramidothioate, S-methyl 1,2,3-benzothiadiazole-7-carbothioate, spiro[2H]-1-benzopyrane-2,1′(3′H)-isobenzofuran-3′-one, 4-[(3,4-dimethoxyphenyl)-3-(4-fluorophenyl)-acryloyl]-morpholine.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Insecticides/Acaricides/Nematicides:

abamectin, acephate, acetamiprid, acrinathrin, alanycarb, aldicarb, aldoxycarb, alpha-cypermethrin, alphamethrin, amitraz, avermectin, AZ 60541, azadirachtin, azamethiphos, azinphos A, azinphos M, azocyclotin, Bacillus popilliae, Bacillus sphaericus, Bacillus subtilis, Bacillus thuringiensis, Baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben, cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine, deltamethrin, demeton M, demeton S, demeton-S-methyl, diafenthiuron, diazinon, dichlorvos, dicofol, diflubenzuron, dimethoate, dimethylvinphos, diofenolan, disulfoton, docusat-sodium, dofenapyn, eflusilanate, emamectin, empenthrin, endosulfan, Entomopfthora spp., esfenvalerate, ethiofencarb, ethion, ethoprophos, etofenprox, etoxazole, etrimfos, fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb, granulosis viruses, halofenozide, HCH, heptenophos, hexaflumuron, hexythiazox, hydroprene, imidacloprid, indoxacarb, isazofos, isofenphos, isoxathion, ivermectin, nuclear polyhedrosis viruses, lambda-cyhalothrin, lufenuron, malathion, mecarbam, metaldehyde, methamidophos, metharhizium anisopliae, metharhizium flavoviride, methidathion, methiocarb, methoprene, methomyl, methoxyfenozide, metolcarb, metoxadiazone, mevinphos, milbemectin, milbemycin, monocrotophos, naled, nitenpyram, nithiazine, novaluron, omethoate, oxamyl, oxydemethon M, Paecilomyces fumosoroseus, parathion A, parathion M, permethrin, phenthoate, phorate, phosalone, phosmet, phosphamidon, phoxim, pirimicarb, pirimiphos A, pirimiphos M, profenofos, promecarb, propargite, propoxur, prothiofos, prothoate, pymetrozine, pyraclofos, pyresmethrin, pyrethrum, pyridaben, pyridathion, pyrimidifen, pyriproxyfen, quinalphos, ribavirin, salithion, sebufos, silafluofen, spinosad, spirodiclofen, sulfotep, sulprofos, tau-fluvalinate, tebufenozide, tebufenpyrad, tebupirimiphos, teflubenzuron, tefluthrin, temephos, temivinphos, terbufos, tetrachlorvinphos, tetradifon, thetacypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazurone, trichlophenidine, trichlorfon, triflumuron, trimethacarb, vamidothion, vaniliprole, Verticillium lecanii, YI 5302, zeta-cypermethrin, zolaprofos, (1R-cis)-[5-(phenylmethyl)-3-furanyl]-methyl 3-[(dihydro-2-oxo-3(2H)-furanylidene)-methyl]-2,2-dimethylcyclopropanecarboxylate, (3-phenoxyphenyl)-methyl 2,2,3,3-tetramethylcyclopropanecarboxylate, 1 -[(2-chloro-5-thiazolyl)methyl]tetrahydro-3,5-dimethyl-N-nitro-1,3,5-triazine-2(1H)-imine, 2-(2-chloro-6-fluorophenyl)-4-[4-(1,1-dimethylethyl)phenyl]-4,5-dihydro-oxazole, 2-(acetyloxy)-3-dodecyl-1,4-naphthalenedione, 2-chloro-N-[[[4-(1-phenylethoxy)-phenyl]-amino]-carbonyl]-benzamide, 2-chloro-N-[[[4-(2,2-dichloro-1,1-difluoroethoxy)-phenyl]-amino]-carbonyl]-benzamide, 3-methylphenyl propylcarbamate, 4-[4-(4-ethoxyphenyl)-4-methylpentyl]-1-fluoro-2-phenoxy-benzene, 4-chloro-2-(1,1-dimethylethyl)-5-[[2-(2,6-dimethyl-4-phenoxyphenoxy)ethyl]thio]-3(2H)-pyridazinone, 4-chloro-2-(2-chloro-2-methylpropyl)-5-[(6-iodo-3-pyridinyl)methoxy]-3(2H)-pyridazinone, 4-chloro-5-[(6-chloro-3-pyridinyl)methoxy]-2-(3,4-dichlorophenyl)-3(2H)-pyridazinone, Bacillus thuringiensis strain EG-2348, [2-benzoyl-1-(1,1 -dimethylethyl)-hydrazinobenzoic acid, 2,2-dimethyl-3-(2,4-dichlorophenyl)-2-oxo-1-oxaspiro[4.5]dec-3-en-4-yl butanoate, [3-[(6-chloro-3-pyridinyl)methyl]-2-thiazolidinylidene]-cyanamide, dihydro-2-(nitromethylene)-2H-1,3-thiazine-3(4H)-carboxaldehyde, ethyl [2-[[1,6-dihydro-6-oxo-1-(phenyhnethyl)-4-pyridazinyl]oxy]ethyl]-carbamate, N-(3,4,4-trifluoro-1-oxo-3-butenyl)-glycine, N-(4-chlorophenyl)-3-[4-(difluoromethoxy)phenyl]-4,5-dihydro-4-phenyl-1H-pyrazole-1-carboxamide, N-[(2-chloro-5-thiazolyl)methyl]-N′-methyl-N″-nitro-guanidine, N-methyl-N′-(1-methyl-2-propenyl)-1,2-hydrazinedicarbothioamide, N-methyl-N′-2-propenyl-1,2-hydrazinedicarbothioamide, O,O-diethyl [2-(dipropylamino)-2-oxoethyl]-ethylphosphoramidothioate, N-cyanomethyl-4-trifluoromethyl-nicotinamide, 3,5-dichloro-1-(3,3-dichloro-2-propenyloxy)-4-[3-(5-trifluoromethylpyridin-2-yloxy)propoxy]-benzene.

A mixture with other known active compounds, such as herbicides, or with fertilizers and growth regulators is also possible.

The active compounds according to the invention can furthermore be present when used as insecticides in their commercially available formulations and in the use forms prepared from these formulations, as a mixture with synergistic agents. Synergistic agents are compounds which increase the action of the active compounds, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as a good stability to alkali on limed substrates.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetic Modified Organisms), and parts thereof a retreated. The term “parts” or “parts of plants” or “plant parts” has been explained above.

Particularly preferably, plants of the plant cultivars which are in each case commercially available or in use are treated according to the invention. Plant cultivars are to be understood as meaning plants having novel properties (“traits”) which have been obtained by conventional breeding, by mutagenesis or by recombinant DNA techniques. This can be varieties, bio- and genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageously useful traits to these plants. Examples of such traits are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapevines), and particular emphasis is given to maize, soya beans, potatoes, cotton and oilseed rape. Traits that are emphasized are in particular increased defence of the plants against insects by toxins formed in the plants, in particular those formed by the genetic material from Bacillus thuringiensis (for example by the genes CryIA(a), CryLA(b), CryIA(c), CryIIA, CryIIIA, CryIIIB2, Cry9c, Cry2Ab, Cry3Bb and CryIF and also combinations thereof) in the plants (hereinbelow referred to as “Bt plants”). Traits that are also particularly emphasized are the increased defence of the plants to fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulfonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulfonylureas, for example maize). Herbicide-resistant plants (plants bred in a conventional manner for herbicide tolerance) which may be mentioned also include the varieties sold under the name Clearfield® (for example maize). Of course, these statements also apply to plant cultivars having these or still-to-be-developed genetic traits, which plants will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants.

Particular emphasis is given to the treatment of plants with the compounds or the mixtures specifically mentioned in the present text.

The active compounds according to the invention act not only against plant, hygiene and stored product pests, but also in the veterinary medicine sector against animal parasites (ectoparasites), such as hard ticks, soft ticks, mange mites, leaf mites, flies (biting and licking), parasitic fly larvae, lice, hair lice, feather lice and fleas. These parasites include:
From the order of the Anoplurida, for example, Haematopinus spp., Linognathus spp., Pediculus spp., Phtirus spp. and Solenopotes spp.
From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.
From the order Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
From the order of the Siphonapterida, for example, Pulex spp., Ctenocephalides spp., Xenopsylla spp. and Ceratophyllus spp.
From the order of the Heteropterida, for example, Cimex spp., Triatoma spp., Rhodnius spp. and Panstrongylus spp.
From the order of the Blattarida, for example, Blatta orientalis, Periplaneta americana, Blattela germanica and Supella spp.
From the subclass of the Acaria (Acarida) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp. and Varroa spp.
From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injections (intramuscular, subcutaneous, intravenous, intraperitoneal and the like), implants, by nasal administration, by dermal use in the form, for example, of dipping or bathing, spraying, pouring on and spotting on, washing and powdering, and also with the aid of molded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100 to 10 000-fold dilution, or they can be used as a chemical bath.

It has furthermore been found that the compounds according to the invention have a strong insecticidal action against insects which destroy industrial materials.

The following insects may be mentioned as examples and as being preferred—but without any limitation:

Beetles, such as Hylotrupes bajulus, Chlorophorus pilosis, Anobium punctatum, Xestobium rufovillosum, Ptilinus pecticomis, Dendrobium pertinex, Emobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec. and Dinoderus minutus.

Hymenoptera, such as Sirex juvencus, Urocerus gigas, Urocerus gigas taignus and Urocerus augur.

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus.

Bristletails, such as Lepisma saccharina.

Industrial materials in the present context are to be understood as meaning non-living materials, such as, preferably, plastics, adhesives, sizes, papers and cards, leather, wood and processed wood products and coating compositions.

Wood and processed wood products are materials to be protected, especially preferably, from insect infestation.

Wood and processed wood products which can be protected by the agent according to the invention or mixtures comprising this are to be understood as meaning, for example:

building timber, wooden beams, railway sleepers, bridge components, boat jetties, wooden vehicles, boxes, pallets, containers, telegraph poles, wood panelling, wooden windows and doors, plywood, chipboard, joinery or wooden products which are used quite generally in house-building or in building joinery.

The active compounds can be used as such, in the form of concentrates or in generally customary formulations, such as powders, granules, solutions, suspensions, emulsions or pastes.

The formulations mentioned can be prepared in a manner known per se, for example by mixing the active compounds with at least one solvent or diluent, emulsifier, dispersing agent and/or binder or fixing agent, a water repellent, if appropriate siccatives and UV stabilizers and if appropriate dyes and pigments, and also other processing auxiliaries.

The insecticidal compositions or concentrates used for the preservation of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by a series of tests. In general, however, it is sufficient to employ 0.0001 to 20% by weight, preferably 0.001 to 10% by weight, of the active compound, based on the material to be preserved.

Solvents and/or diluents which are used are an organic chemical solvent or solvent mixture and/or an oily or oil-like organic chemical solvent or solvent mixture of low volatility and/or a polar organic chemical solvent or solvent mixture and/or water, and if appropriate an emulsifier and/or wetting agent.

Organic chemical solvents which are preferably used are oily or oil-like solvents having an evaporation number above 35 and a flashpoint above 30° C., preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkylbenzene.

Mineral oils having a boiling range from 170 to 220° C., white spirit having a boiling range from 170 to 220° C., spindle oil having a boiling range from 250 to 350° C., petroleum and aromatics having a boiling range from 160 to 280° C., turpentine oil and the like, are advantageously employed.

In a preferred embodiment, liquid aliphatic hydrocarbons having a boiling range from 180 to 210° C. or high-boiling mixtures of aromatic and aliphatic hydrocarbons having a boiling range from 180 to 220° C. and/or spindle oil and/or monochloronaphthalene, preferably α-monochloronaphthalene, are used.

The organic oily or oil-like solvents of low volatility which have an evaporation number above 35 and a flashpoint above 30° C., preferably above 45° C., can be replaced in part by organic chemical solvents of high or medium volatility, providing that the solvent mixture likewise has an evaporation number above 35 and a flashpoint above 30° C., preferably above 45° C., and that the insecticide/fungicide mixture is soluble or emulsifiable in this solvent mixture.

According to a preferred embodiment, some of the organic chemical solvent or solvent mixture is replaced by an aliphatic polar organic chemical solvent or solvent mixture. Aliphatic organic chemical solvents containing hydroxyl and/or ester and/or ether groups, such as, for example, glycol ethers, esters or the like, are preferably used.

Organic chemical binders which are used in the context of the present invention are the synthetic resins and/or binding drying oils which are known per se, are water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-cumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odor correctants and inhibitors or anticorrosive agents and the like which are known per se can additionally be employed.

It is preferred according to the invention for the composition or concentrate to comprise, as the organic chemical binder, at least one alkyd resin or modified alkyd resin and/or one drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phthalic acid esters, such as dibutyl, dioctyl or benzyl butyl phthalate, phosphoric acid esters, such as tributyl phosphate, adipic acid esters, such as di-(2-ethylhexyl) adipate, stearates, such as butyl stearate or amyl stearate, oleates, such as butyl oleate, glycerol ethers or higher molecular weight glycol ethers, glycerol esters and p-toluenesulfonic acid esters.

Fixing agents are based chemically on polyvinyl alkyl ethers, such as, for example, polyvinyl methyl ether or ketones, such as benzophenone or ethylenebenzophenone.

Possible solvents or diluents are, in particular, also water, if appropriate as a mixture with one or more of the abovementioned organic chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Especially preferred mixing partners which may be mentioned are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyphenoxide and triflumuron, and also fungicides, such as epoxyconazole, hexaconazole, azaconazole, propiconazole, tebuconazole, cyproconazole, metconazole, imazalil, dichlorfluanid, tolylfluanid, 3-iodo-2-propinyl-butyl carbamate, N-octyl-isothiazolin-3-one and 4,5-dichloro-N-octylisothiazolin-3 -one.

The compounds according to the invention can at the same time be employed for protecting objects which come into contact with salt water or brackish water, in particular hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis-(trialkyltin) sulfides, tri-n-butyltin laurate, tri-n-butyltin chloride, copper(I) oxide, triethyltin chloride, tri-n-butyl-(2-phenyl-4-chlorophenoxy)tin, tributyltin oxide, molybdenum disulfide, antimony oxide, polymeric butyl titanate, phenyl-(bispyridine)-bismuth chloride, tri-n-butyltin fluoride, manganese ethylenebisthio-carbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combination with the antifouling compositions according to the invention are:

algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

fungicides such as benzo[b]thiophenecarboxylic acid cyclohexylamide S,S-dioxide, dichlofluanid, fluor-folpet, 3-iodo-2-propinyl butylcarbamate, tolylfluanid and azoles such as azaconazole, cyproconazole, epoxyconazole, hexaconazole, metconazole, propiconazole and tebuconazole;

molluscicides such as fentin acetate, metaldehyde, methiocarb, niclosamid, thiodicarb and trimethacarb; or conventional antifouling active compounds such as 4,5-dichloro-2-octyl-4-isothiazolin-3-one, diiodomethylparatryl sulfone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulfonyl)-pyridine, 2,4,5,6-tetrachloroisophthalonitrile, tetra-methylthiuram disulfide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as rosin to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:
From the order of the Scorpionidea, for example, Buthus occitanus.
From the order of the Acarina, for example, Argas persicus, Argas reflexus, Bryobia ssp., Dermanyssus gallinae, Glyciphagus domesticus, Omithodorus moubat, Rhipicephalus sanguineus, Trombicula alfreddugesi, Neutrombicula autumnalis, Dermatophagoides pteronissimus and Dermatophagoides forinae.
From the order of the Araneae, for example, Aviculariidae and Araneidae.
From the order of the Opiliones, for example, Pseudoscorpiones chelifer, Pseudoscorpiones cheiridium and Opiliones phalangium.
From the order of the Isopoda, for example, Oniscus asellus and Porcellio scaber.
From the order of the Diplopoda, for example, Blaniulus guttulatus and Polydesmus spp.
From the order of the Chilopoda, for example, Geophilus spp.
From the order of the Zygentoma, for example, Ctenolepisma spp., Lepisma saccharina and Lepismodes inquilinus.
From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa and Supella longipalpa.
From the order of the Saltatoria, for example, Acheta domesticus.
From the order of the Dermaptera, for example, Forficula auricularia.
From the order of the Isoptera, for example, Kalotermes spp. and Reticulitermes spp.
From the order of the Psocoptera, for example, Lepinatus spp. and Liposcelis spp.
From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais and Stegobium paniceum.
From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga camaria, Simulium spp., Stomoxys calcitrans and Tipula paludosa.
From the order of the Lepidoptera, for example, Achroia grisella, Galleria mellonella, Plodia interpunctella, Tinea cloacella, Tinea pellionella and Tineola bisselliella.
From the order of the Siphonaptera, for example, Ctenocephalides canis, Ctenocephalides felis, Pulex irritans, Tunga penetrans and Xenopsylla cheopis.
From the order of the Hymenoptera, for example, Camponotus herculeanus, Lasius fuliginosus, Lasius niger, Lasius umbratus, Monomorium pharaonis, Paravespula spp. and Tetramorium caespitum.
From the order of the Anoplura, for example, Pediculus humanus capitis, Pediculus humanus corporis and Phthirus pubis.
From the order of the Heteroptera, for example, Cimex hemipterus, Cimex lectularius, Rhodinus prolixus and Triatoma infestans.

They are used in the household insecticides sector alone or in combination with other suitable active compounds such as phosphoric esters, carbamates, pyrethroids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The preparation and the use of the compounds according to the invention is shown in the examples below.

### PREPARATION EXAMPLES

### Example 1

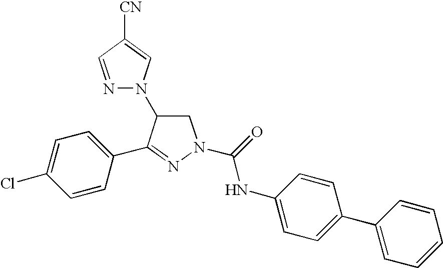 (Process a)

At 70° C., 0.72 g (3.7 mmol) of 4-phenylphenyl isocyanate is added to a mixture of 1 g (3.7 mmol) of 3 -(4-chlorophenyl)-4-(4-cyanopyrazol-1-yl)-4,5-dihydro-1H-pyrazole, 0.1 ml of triethylamine and 80 ml of methyl tert-amyl ether. The mixture is stirred at 70° C. for 15 minutes and then allowed to slowly cool to room temperature. The precipitated product is filtered off with suction and washed with a little methyl tert-butyl ether.

This gives 1.2 g (69% of theory) of N-(4-phenyl)-3-(4-chlorophenyl)-4-(4-cyanopyrazol-1-yl)-4,5-dihydro-1-pyrazolecarboxanilide of logP (pH2)=4.11.
Preparation of the Starting Material

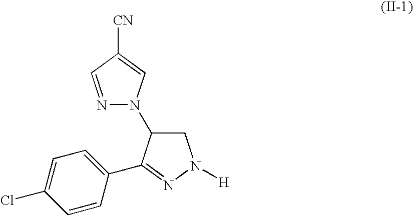

At room temperature, 2.16 (0.021 mol) of bisdimethylaminomethane are added to a solution of 5 g (0.02 mol) of 2-(4-cyanopyrazol-1-yl)-4′-chloroacetophenone in 50 ml of methylene chloride, and the mixture is boiled under reflux for 18 hours. The solvent is then distilled off under reduced pressure and the residue is dissolved in 50 ml of ethanol. After addition of 1.13 g (0.0226 mol) of hydrazine hydrate, the reaction mixture is stirred at 30° C. for 3 hours. The precipitated product is filtered off with suction and washed with a little cold ethanol and with water.

This gives 3.3 g (61% of theory) of 3-(4-chlorophenyl)-4-(4-cyanopyrazol-1-yl)-4,5-dihydro-1H-pyrazole of logP (pH2)=2.11.
Preparation of the Precursor

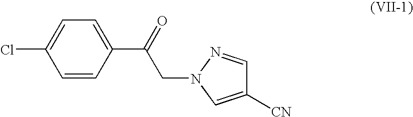

A mixture of 9.3 g (0.04 mol) of 2-bromo-4′-chloroacetophenone, 3.9 g (0.042 mol) of 4-cyanopyrazole (preparation see JP H59-196868), 6.1 g (0.044 mol) of potassium carbonate and 50 ml of acetonitrile is stirred at room temperature overnight. About 200 ml of water are then added to the reaction mixture and the precipitated product is filtered off with suction and washed with water.

This gives 9.5 g (97% of theory) of 2-(4-cyanopyrazol-1-yl)-4′-chloroacetophenone of logP (pH2)=2.20.

### Example 2

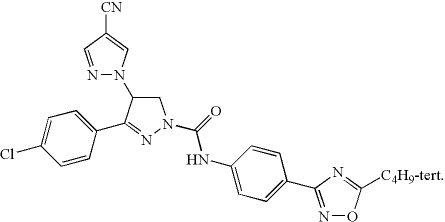 (Process c)

At 0° C., a solution of 1.1 g (5 mmol) of 4-(5-tert-butyl-[1,2,4]oxadiazol-3-yl)aniline and 0.7 ml (5 mmol) of triethylamine in 25 ml of methylene chloride is added dropwise to a solution of 0.5 g (1.7 mmol) of triphosgene (bistrichloromethyl carbonate) in 50 ml of methylene chloride, and the mixture is stirred at room temperature for half an hour. A solution of 1.36 (5 mmol) of 3-(4-chlorophenyl)-4-(4-cyanopyrazol-1-yl)-4,5-dihydro-1H-pyrazole and 0.7 ml (5 mmol) of triethylamine in 25 ml of methylene chloride is then added dropwise. The mixture is stirred at room temperature for 18 hours and then washed twice with in each case 100 ml of water. The organic phase is dried over sodium sulfate and concentrated under reduced pressure. The residue is triturated with ethanol, filtered off with suction and washed with ethanol.

This gives 0.6 g (23% of theory) of N-[4-(5-tert-butyl[1.2.4]oxadiazol-3-yl)-phenyl]-3-(4-chlorophenyl)-4-(4-cyanopyrazol-1-yl)-4,5-dihydro-1-pyrazolecarboxamide of logP (pH2)=4.33.
Preparation of the Starting Material

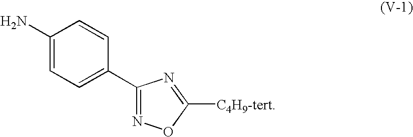

A mixture of 4.5 g (0.03 mol) of 4-aminobenzamidoxime, 3.5 g (0.03 mol) of methyl pivalate, 50 ml of ethanol, 1 ml of a 28% strength sodium methoxide solution in methanol and 2 g of molecular sieve 3 A is boiled under reflux for 18 hours. The reaction mixture is then filtered and the filtrate is concentrated under reduced pressure. Water is added to the residue, and the precipitated crystals are then filtered off with suction. The crude product is purified by silica gel chromatography (methylene chloride/diethyl ether 3:1).

This gives 2.1 g (33% of theory) of 4-(5-tert-butyl[1,2,4]oxadiazol-3-yl)aniline in the form of colorless crystals of logP (pH2) of 2.31.

### Example 3

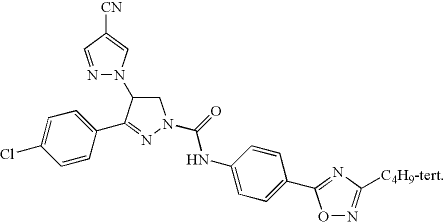 (Process c)

At 0° C., a solution of 1.1 g (5 mmol) of 4-(3-tert-butyl[1,2,4]oxadiazol-5-yl)aniline and 0.7 ml (5 mmol) of triethylamine in 25 ml methylene chloride is added dropwise to a solution of 0.5 g ( 1.7 mmol) of triphosgene (bistrichloromethyl carbonate) in 50 ml of methylene chloride, and the mixture is stirred at room temperature for half an hour. A solution of 1.36 g (5 mmol) of 3-(4-chlorophenyl)-4-(4-cyanopyrazol-1-yl)-4,5-dihydro-1H-pyrazole and 0.7 ml (5 mmol) of triethylamine in 25 ml of methylene chloride is then added dropwise. The mixture is stirred at room temperature for 18 hours and then washed twice with in each case 100 ml of water. The organic phase is dried over sodium sulfate and concentrated under reduced pressure. The residue is triturated with ethanol, filtered off with suction and washed with ethanol.

This gives 0.95 g (37% of theory) of N-[4-(3-tert-butyl[1,2,4]oxadiazol-5-yl)-phenyl]-3-(4-chlorophenyl)-4-(4-cyanopyrazol-1-yl)-4,5-dihydro-1-pyrazolecarbox-amide of logP (pH2)=4.38.
Preparation of the Starting Material

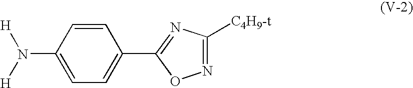

A mixture of 6 g (0.04 mol) of methyl 4-aminobenzoate, 5.8 g (0.05 mol) of pival-amidoxime, 50 ml of ethanol, 1 ml of a 28% strength sodium methoxide solution in methanol and 2 g of molecular sieve 3A is boiled under reflux for 18 hours. Most of the solvent is then distilled off under reduced pressure, the residue is cooled in an ice bath, a little water is added and the precipitated crystals are then filtered off with suction. The crystals are washed with water and the residue is dissolved in a solution of 4 g of potassium hydroxide in 50 ml of methanol. The solution is allowed to stand at room temperature for 3 days and the solvent is then distilled off under reduced pressure. The residue is triturated with water and the precipitated product is filtered off with suction and washed with water.

This gives 2.95 g (34% of theory) of 4-(3-tert-butyl[1,2,4]oxadiazol-5-yl)aniline in the form of colorless crystals of logP (pH2) of 2.54.

### Example 4

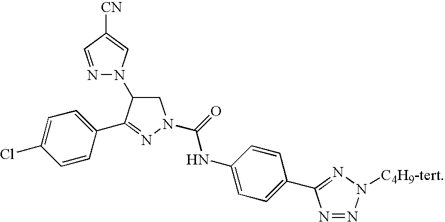 (Process c)

At 0° C., a solution of 0.64 g (3 mmol) of 4-(2-tert-butyltetrazol-5-yl)aniline and 0.4 ml (3 mmol) of triethylamine in 25 ml of methylene chloride is added dropwise to a solution of 0.35 g (1.2 mmol) of triphosgene (bistrichloromethyl carbonate) in 30 ml of methylene chloride, and the mixture is stirred at room temperature for half an hour. A solution of 0.8 g (3 mmol) of 3-(4-chlorophenyl)-4-(4-cyanopyrazol-1-yl)-4,5-dihydro-1H-pyrazole and 0.4 ml (3 mmol) of triethylamine in 25 ml of methylene chloride is then added dropwise. The mixture is stirred at room temperature for 18 hours and then washed twice with in each case 100 ml of water. The organic phase is dried over sodium sulfate and concentrated under reduced pressure. The residue is purified by silica gel chromatography (methylene chloride/diethyl ether 1:1).

This gives 0.3 g (19.5% of theory) of N-[4-(2-tert-butyltetrazol-5-yl)phenyl]-3-(4-chlorophenyl)-4-(4-cyanopyrazol-1-yl)-4,5-dihydro-1-pyrazolecarboxamide of logP (pH2)=3.91.
Preparation of the Starting Material

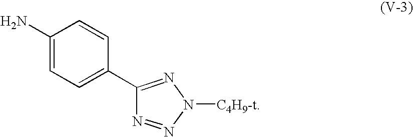

3.8 ml of concentrated sulfuric acid are added to a mixture of 15 g (0.093 mol) of 5-(4-aminophenyl)tetrazole, 28 ml of tert-butanol and 80 ml of trifluoroacetic acid. The reaction mixture is stirred for 16 hours and the solvent is then distilled off under reduced pressure and saturated sodium bicarbonate solution is added to the residue. The product is extracted with ethyl acetate. The organic phase is dried over sodium sulfate and the solvent is then distilled off under reduced pressure.

What remains are 14.8 g (73% of theory) of 4-(2-tert-butyltetrazol-5-yl)aniline in the form of beige crystals of logP (pH2)=1.69.

### Example 5

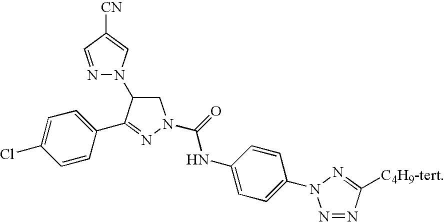 (Process c)

At 0° C., a solution of 1.2g (4.6 mmol) of 3-(4-chlorophenyl)-4-(4-cyanopyrazol-1-yl)-4,5-dihydro-1H-pyrazole and 0.65 ml (4.6 mmol) of triethylamine in 25 ml of methylene chloride is added dropwise to a solution of 0.45 g (1.54 mmol) of triphosgene (bistrichloromethyl carbonate) in 30 ml of methylene chloride, and the mixture is stirred at room temperature for half an hour. A solution of 1 g (4.6 mmol) of 4-(5-tert-butyltetrazol-2-yl)aniline and 0.65 ml (4.6 mmol) of triethylamine in 25 ml of methylene chloride is then added dropwise. The mixture is stirred at room temperature for 18 hours and then washed twice with in each case 100 ml of water. The organic phase is dried over sodium sulfate and concentrated under reduced pressure. The residue is purified by silica gel chromatography (methylene chloride/diethyl ether 1:1).

This gives 0.3 g (13% of theory) of N-[4-(5-tert-butyltetrazol-2-yl)phenyl]-3-(4-chlorophenyl)-4-(4-cyanopyrazol-1-yl)-4,5-dihydro-1-pyrazolecarboxamide of logP (pH2)=4.27.
Preparation of the Starting Material

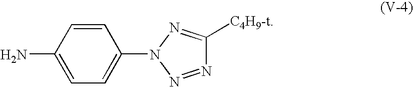

At 75° C., 8.7 ml of hydrazine hydrate are added dropwise over a period of two hours to a mixture of 12.7 g (0.051 mol) of 4-(5-tert-butyltetrazol-2-yl)nitrobenzene, 1.5 g of activated carbon, 0.3 g of iron(III) chloride, 20 ml of methanol and 100 ml of tetrahydrofuran. The mixture is then stirred at 75° C. overnight, cooled and filtered off with suction through kieselguhr, and the filter cake is washed thoroughly with ethyl acetate. The filtrate is washed with sodium chloride solution, dried and concentrated under reduced pressure.

This gives 15 g (quantitative) of 4-(5-tert-butyltetrazol-2-yl)aniline of logP (pH2)=2.27.
Preparation of the Precursor

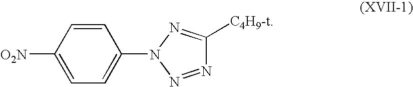

A mixture of 9.7 g (0.07 mol) of 4-fluoronitrobenzene, 8.7 g (0.7 mol) of 5-tert-butyltetrazole, 10.6 g (0.077 mol) of potassium carbonate and 100 ml of dimethylformamide is stirred at 75° C. overnight. The reaction mixture is then cooled to room temperature and poured into 600 g of ice-water. The precipitated product is filtered off with suction and washed with water.

This gives 12.7 g (73% of theory) of 4-(5-tert-butyltetrazol-2-yl)nitrobenzene of logP (pH2)=2.20.

Analogously to Examples 1 to 5 and in accordance with the general statements on the preparation, it is possible to obtain the compounds of the formula (I) listed in Table 1 below:

TABLE 1

| | |
|---|---|
| | (I) |
| 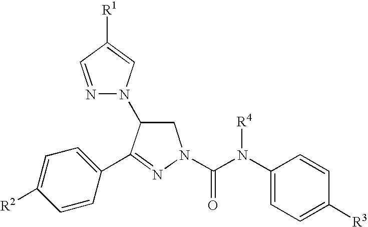 | |

| No. | R¹ | R² | R³ | R⁴ | logP (pH2) |
|---|---|---|---|---|---|
| 6 | CN | Cl | 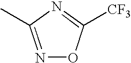 | H | 4.31 |
| 7 | CN | Cl | 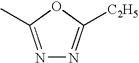 | H | 2.93 |
| 8 | CN | Cl | 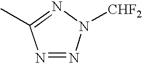 | H | 3.49 |
| 9 | CN | Cl | 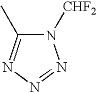 | H | 3.10 |
| 10 | CN | Cl | 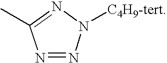 | CH₂CN | 3.59 |
| 11 | CN | Cl | 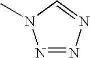 | H | 2.61 |
| 12 | CN | Cl | 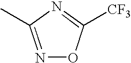 | CH₂CN | 4.00 |

Analogously to Examples 1 to 5 and in accordance with the general statements on the preparation, it is possible to obtain the compounds of the formula (II) listed in Table 2 below:

TABLE 2

| | |
|---|---|
| | (II) |
| 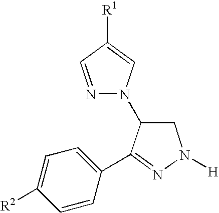 | |

| No. | R¹ | R² | logP (pH2) |
|---|---|---|---|
| 11-2 | CN | Br | 2.20 |
| 11-3 | CN | I | 2.33 |
| 11-4 | CN | SCF₃ | 2.78 |

Analogously to Example 1 and in accordance with the general statements on the preparation, it is possible to obtain the precursors of the formula (VII) listed in Table 3:

TABLE 3

| | |
|---|---|
| | (VII) |
| 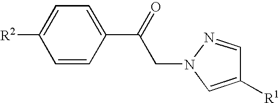 | |

| No. | R¹ | R² | logP (pH2) |
|---|---|---|---|
| (VII)-2 | CN | Br | 2.31 |
| (VII)-3 | CN | I | 2.47 |
| (VII)-4 | CN | SCF₃ | 2.91 |

The logP values given in the Preparation Examples and the tables above are determined in accordance with EEC Directive 79/831 Annex V.A8 by HPLC (High Performance Liquid Chromatography) on a reversed-phase column (C 18). Temperature: 43° C.

The determination is carried out in the acidic range at pH 2.3 using the mobile phases 0.1% aqueous phosphoric acid and acetonitrile; linear gradient from 10% acetonitrile to 90% acetonitrile.

Calibration is carried out using unbranched alkan-2-ones (having 3 to 16 carbon atoms) with known logP values (determination of the logP values by the retention times using linear interpolation between two successive alkanones).

### Use Examples

### Example A

Phaedon Larvae Test

Solvent: 7 parts by weight of dimethylformamide

Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with larvae of the mustard beetle (Phaedon cochleariae) while the leaves are still moist.

After the desired period of time, the kill in % is determined. 100% means that all beetle larvae have been killed; 0% means that none of the beetle larvae have been killed.

In this test, at an exemplary active compound concentration of 500 ppm, for example, the compounds 2, 4, 6, 7 and 9 of the Preparation Examples show a kill of 100% after 7 days.

### Example B

Spodoptera Frugiperda Test

Solvent: 7 parts by weight of dimethylformamide

Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the army worm (Spodoptera frugiperda) while the leaves are still moist.

After the desired period of time, the kill in % is determined. 100% means that all caterpillars have been killed; 0% means that none of the caterpillars have been killed.

In this test, at an exemplary active compound concentration of 500 ppm, for example, the compounds 1, 2, 3, 4 and 9 of the Preparation Examples show a kill of 100% after 7 days.

### Example C

Diabrotica Balteata Test (Larvae in Soil)

Critical concentration test/soil insects—treatment of transgenic plants

Solvent: 7 parts by weight of dimethylformamide

Emulsifier: 1 part by weight of alkylaryl polyglycol ether

To produce a suitable preparation of active compound, 1 part by weight of active compound is mixed with the stated amount of solvent, the stated amount of emulsifier is added and the concentrate is diluted with water to the desired concentration.

The preparation of active compound is poured onto the soil. Here, the concentration of active compound in the preparation is virtually immaterial, only the amount by weight of active compound per volume unit of soil, which is stated in ppm (mg/l), matters. The soil is filled in 0.25 1 pots, and these are allowed to stand at 20° C.

Immediately after the preparation, 5 pregerminated maize corns of the cultivar YIELD GUARD (trade mark of Monsanto Comp., USA) are placed into each pot. After 2 days, the appropriate test insects are placed into the treated soil. After a further 7 days, the efficacy of the active compound is determined by counting the maize plants that have emerged (1 plant=20% activity).

### Example D

Heliothis Virescens Test (Treatment of Transgenic Plants)

Solvent: 7 parts by weight of dimethylformamide

Emulsifier: 1 part by weight of alkylaryl polyglycol ether

To produce a suitable preparation of active compound, 1 part by weight of active compound is mixed with the stated amount of solvent and the stated amount of emulsifier, and the concentrate is diluted with water to the desired concentration.

Soya bean shoots (Glycine max) of the cultivar Roundup Ready (trade mark of Monsanto Comp. USA) are treated by being dipped into the preparation of active compound of the desired concentration and are populated with the tobacco budworm Heliothis virescens while the leaves are still moist.

After the desired period of time, the kill in % is determined. 100% means that all caterpillars have been killed; 0% means that none of the caterpillars have been killed.

1-14. (canceled)

15. A substituted pyrazoline of formula (I) 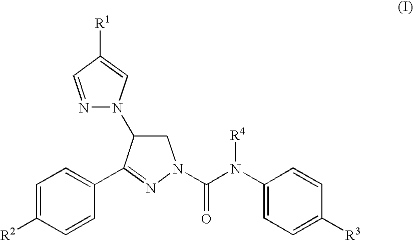
or isomers or isomer mixtures thereof,
in which
R¹ represents halogen or cyano,
R² represents halogen, haloalkyl, alkoxy, haloalkoxy, alkylthio, haloalkylthio, alkylsulfonyl, haloalkylsulfinyl, haloalkylsulfonyl, or cyano,
R³ represents optionally substituted aryl or optionally substituted hetaryl, and
R⁴ represents hydrogen, cyanomethyl, or alkoxycarbonyl.

16. A substituted pyrazoline of formula (I) as claimed in claim 15 in which
R¹ represents fluorine, chlorine, bromine, iodine, or cyano,
R² represents fluorine, chlorine, bromine, iodine, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfinyl, C₁-C₄-haloalkylsulfonyl, or cyano,
R³ represents aryl that is optionally mono- or polysubstituted by identical or different substituents selected from the group consisting of halogen, alkyl, alkoxy, alkylthio, alkylsulfonyl, haloalkyl, haloalkoxy, haloalkylthio, haloalkylsulfonyl and cyano; represents optionally monosubstituted oxadiazolyl or thiadiazolyl, wherein the substituent is optionally substituted alkyl, optionally substituted alkoxy, optionally substituted alkylthio, optionally substituted aryl, or optionally substituted arylalkyl; or represents optionally monosubstituted tetrazolyl, wherein the substituent is optionally substituted alkyl, optionally substituted alkylthio or alkylsulfonyl, optionally substituted aryl or arylalkyl, or optionally substituted cycloalkyl, and
R⁴ represents hydrogen, cyanomethyl or C₁-C₄-alkoxycarbonyl.

17. A substituted pyrazoline of formula (I) as claimed in claim 15 in which
R¹ represents chlorine, bromine, iodine, or cyano,
R² represents fluorine, chlorine, bromine, iodine, cyano, C₁-C₂-alkylthio, or C₁-C₂-alkylsulfonyl; or represents C₁-C₂-haloalkyl, C₁-C₂-haloalkoxy, C₁-C₂-haloalkylthio, or C₁-C₂-haloalkylsulfonyl having in each case 1 to 5 identical or different halogen atoms selected from the group consisting of fluorine, chlorine, and bromine,
R³ represents phenyl that is optionally mono- to trisubstituted by identical or different substituents, wherein the substituents are fluorine, chlorine, bromine, iodine, cyano, C₁-C₄-alkyl, C₁-C₄-alkoxy, C₁-C₄-alkylthio, C₁-C₄-alkylsulfonyl, or C₁ -C₄-haloalkyl, C₁-C₄-haloalkoxy, C₁-C₄-haloalkylthio, or C₁-C₄-haloalkylsulfonyl having in each case 1 to 5 identical or different halogen atoms selected from the group consisting of fluorine, chlorine, and bromine; represents optionally monosubstituted oxadiazolyl or thiadiazolyl, wherein the substituents are 4-alkyl, C₁-C₄-haloalkyl, C₁-C₄-alkoxy, C₁-C₄-haloalkoxy, C₁-C₄-alkylthio, or C₁-C₄-haloalkylthio; represents phenyl or benzyl, each of which is optionally mono- to trisubstituted by identical or different substituents selected from the group consisting of halogen, C₁-C₄-haloalkyl, and C₁-C₄ haloalkoxy; represents optionally substituted tetrazolyl, wherein the substituents are C₁-C₄-alkyl, C₁-C₄-haloalkyl, C₁-C₄-alkylthio, C₁-C₄-alkylsulfonyl; represents phenyl or benzyl, each of which is optionally mono- to trisubstituted by identical or different substituents selected from the group consisting of halogen, C₁-C₄-haloalkyl, and C₁-C₄-haloalkoxy; or represents cyclopentyl or cyclohexyl, each of which is optionally mono- to trisubstituted by identical or different substituents selected from the group consisting of C₁-C₄-alkyl, and
R⁴ represents hydrogen, cyanomethyl or C₁-C₄-alkoxycarbonyl.

18. A substituted pyrazoline of formula (I) as claimed in claim 15 in which
R¹ represents chlorine, bromine, or cyano,
R² represents fluorine, chlorine, bromine, iodine, methylthio, trifluoromethyl, trifluoromethoxy, or trifluoromethylthio,
R³ represents phenyl that is optionally mono- to trisubstituted by identical or different substituents, wherein the substituents are fluorine, chlorine, bromine, iodine, cyano, methyl, methoxy, methylthio, trifluoromethyl, trifluoromethoxy, trifluoromethylthio or trifluoromethylsulfonyl; represents an oxadiazolyl group selected from the group consisting of 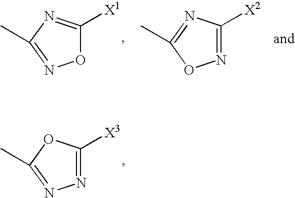
where X¹, X², and X³ independently of one another represent hydrogen, C₁-C₄-alkyl, C₁-C₄-haloalkyl, C₁-C₄-alkylthio, or C₁ -C₄-haloalkylthio; or phenyl or benzyl, each of which is optionally mono- to trisubstituted by identical or different substituents selected from the group consisting of halogen, C₁-C₂-haloalkyl, and C₁ -C₂-haloalkoxy having in each case 1 to 3 identical or different halogen atoms selected from the group consisting of fluorine, chlorine, and bromine; or represents a tetrazolyl group selected from the group consisting of: 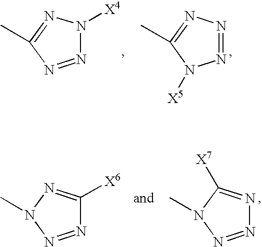
where X⁴, X⁵, X⁶, and X⁷ independently of one another represent hydrogen, C₁-C₄-alkyl, C₁-C₂-haloalkyl having 1 to 3 identical or different halogen atoms selected from the group consisting of fluorine, chlorine, and bromine; C₁-C₄-alkylthio; C₁-C₄-alkylsulfonyl; phenyl or benzyl, each of which is optionally mono- to trisubstituted by identical or different substituents selected from the group consisting of halogen, C₁-C₂-haloalkyl, and C₁-C₂-haloalkoxy having in each case 1 to 3 identical or different halogen atoms selected from the group consisting of fluorine, chlorine; and bromine; or cyclopentyl or cyclohexyl, each of which is optionally mono- to trisubstituted by C₁-C₄-alkyl, and R⁴ represents hydrogen, cyanomethyl, methoxycarbonyl, ethoxycarbonyl, n- or i-propoxycarbonyl, or n-, i-, s-, or t-butoxycarbonyl.

19. A substituted pyrazoline of formula (I) as claimed in claim 15 in which
R¹ represents chlorine or cyano,
R² represents fluorine, chlorine, bromine, iodine, or trifluoromethylthio,
R³ represents phenyl that is optionally mono- or disubstituted by identical or different substituents selected from the group consisting of fluorine, chlorine, trifluoromethyl, trifluoromethoxy, and trifluoromethylthio; represents an oxadiazolyl group selected from the group consisting of: 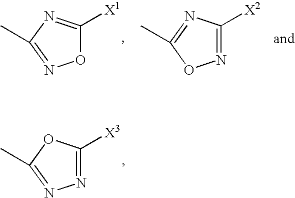
where X¹, X² and X³ independently of one another represent hydrogen, methyl, ethyl, n- or i-propyl, n-, i-, s-, or t-butyl, trifluoromethyl, trifluoromethoxy, or trifluoromethylthio; or phenyl or benzyl, each of which is optionally mono- or disubstituted by identical or different substituents selected from the group consisting of fluorine, chlorine, bromine, trifluoromethyl, and trifluoromethoxy; represents a tetrazolyl group selected from the group consisting of: 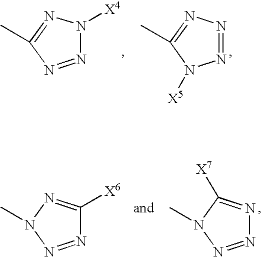
where X⁴, X⁵, X⁶ and X⁷ independently of one another represent hydrogen, methyl, ethyl, n- or i-propyl, n-, i-, s-, or t-butyl, fluoromethyl, difluoromethyl, trifluoromethyl, 1,1-difluoroethyl, 2,2,2-trifluoroethyl, methylthio, ethylthio, methylsulfonyl, or ethylsulfonyl; phenyl or benzyl, each of which is optionally mono- to disubstituted by identical or different substituents selected from the group consisting of fluorine, chlorine, bromine, methyl, methoxy, trifluoromethyl, and trifluoromethoxy; or cyclohexyl that is optionally mono- to disubstituted by methyl, and
R⁴ represents hydrogen or cyanomethyl.

20. A substituted pyrazoline of formula (I) as claimed in claim 15 in which R¹ is cyano.

21. A substituted pyrazoline of formula (I) as claimed in claim 15 in which R² is halogen.

22. A substituted pyrazoline of formula (I) as claimed in claim 15 in which R² is fluorine, chlorine, bromine, or iodine.

23. A substituted pyrazoline of formula (I) as claimed in claim 15 in which R¹ is cyano and R² is chlorine.

24. A substituted pyrazoline of formula (I) as claimed in claim 15 in which R⁴ is hydrogen or cyanomethyl.

25. A process for preparing substituted pyrazolines of formula (I) as claimed in claim 15 comprising
(a) reacting a pyrazoline of formula (II) 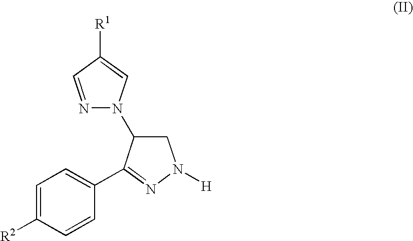
or isomers or isomer mixtures thereof,
in which R¹ and R² are as defined for formula (I) in claim 15, with an isocyanate of formula (III) 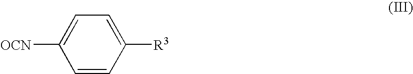
or isomers or isomer mixtures thereof,
in which R³ is as defined for formula (I) in claim 15,
optionally in the presence of a diluent and optionally in the presence of a catalyst,
to form a pyrazoline derivative of formula (la) according to the invention 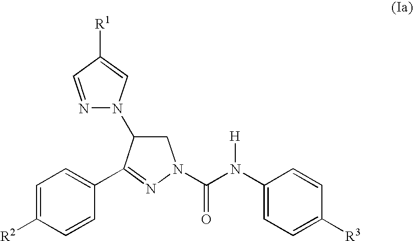
or isomers or isomer mixtures thereof,
in which R¹, R², and R³ are as defined for formula (I) in claim 15, and, optionally,
(b) reacting the pyrazoline derivative of formula (Ia) with a halide of formula (IV)
Hal¹-R⁴ (IV)
or isomers or isomer mixtures thereof,
in which
R⁴ is as defined for formula (I) in claim 15, and
Hal¹ represents halogen,
optionally in the presence of a diluent and optionally in the presence of a base;
or
(c) initially reacting an aniline of formula (V) 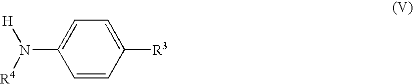
or isomers or isomer mixtures thereof,
in which R³ and R⁴ are as defined for formula (I) in claim 15, with phosgene in the presence of a diluent and optionally in the presence of a base,
to form a carbamoyl chloride of formula (VI) 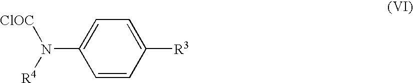
or isomers or isomer mixtures thereof,
in which R³ and R⁴ are as defined for formula (I) in claim 15, and reacting the carbamoyl chloride of formula (VI), directly or after intermediate isolation, with a pyrazoline of formula (II) 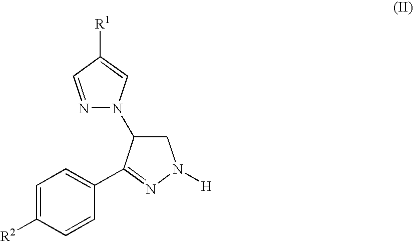
or isomers or isomer mixtures thereof,
in which R¹ and R² are as defined for formula (I) in claim 15, in the presence of a diluent and optionally in the presence of a base.

26. A pesticide comprising one or more compounds of formula (I) as claimed in claim 15 and one or more extenders and/or surfactants.

27. A method of controlling pests comprising allowing an effective amount of a compound of formula (I) as claimed in claim 15 to act on pests and/or their habitat.

28. A process for preparing a pesticide comprising mixing a compound of formula (I) as claimed in claim 15 with one or more extenders and/or surfactants.
